(19) United States
(12) Patent Application Publication
(10) Pub. No.: US 2015/0005874 A1
(43) Pub. Date: Jan. 1, 2015

(54) Atrial Thrombogenic Sealing Pockets for Prosthetic Mitral Valves

(71) Applicant: Tendyne Holdings, Inc., Roseville, MN (US)

(72) Inventors: Robert M. VIDLUND, Forest Lake, MN (US); Zachary J. Tegels, Minneapolis, MN (US); Craig A. Ekvall, Bethel, MN (US)

(73) Assignee: Tendyne Holdings, Inc., Roseville, MN (US)

(21) Appl. No.: 14/260,843

(22) Filed: Apr. 24, 2014

Related U.S. Application Data
(60) Provisional application No. 61/840,313, filed on Jun. 27, 2013.

(51) Int. Cl. A61F 2/24

(52) U.S. Cl. CPC A61F 2/2412; A61F 2/2427
USPC 623/2.14

(57) ABSTRACT

This invention relates to a self-expanding pre-configured compressible transcatheter prosthetic cardiovascular valve that comprises atrial thrombogenic sealing pocket cover mounted on a self-expanding inner wire frame having a leaflet structure comprised of articulating leaflets that define a valve function, said inner wire frame is disposed within a self-expanding annular tissue-covered outer wire frame, said outer wire frame having an articulating collar, forming a multi-component prosthetic valve assembly for anchoring within the mitral valve or triscuspid valve of the heart, and methods for deploying such a valve for treatment of a patient in need thereof.

### CROSS REFERENCE TO RELATED APPLICATIONS

Not applicable

### STATEMENT REGARDING FEDERALLY SPONSORED RESEARCH OR DEVELOPMENT

No federal government funds were used in researching or developing this invention.

### NAMES OF PARTIES TO A JOINT RESEARCH AGREEMENT

Not applicable.

### SEQUENCE LISTING INCLUDED AND INCORPORATED BY REFERENCE HEREIN

Not applicable.

### BACKGROUND

1. Field of the Invention

This invention relates to an improved transcatheter prosthetic heart valve that comprises atrial thrombogenic sealing pockets and mesh covering that is disposed between an inner wire framed leaflet support and an outer self-expanding annular stent that forms a multi-component prosthetic valve assembly that is anchored within the mitral valve or triscuspid valve of the heart.

2. Background of the Invention

Valvular heart disease and specifically aortic and mitral valve disease is a significant health issue in the US Annually approximately 90,000 valve replacements are conducted in the US. Traditional valve replacement surgery, the orthotopic replacement of a heart valve, is an “open heart” surgical procedure. Briefly, the procedure necessitates surgical opening of the thorax, the initiation of extra-corporeal circulation with a heart-lung machine, stopping and opening the heart, excision and replacement of the diseased valve, and re-starting of the heart. While valve replacement surgery typically carries a 1-4% mortality risk in otherwise healthy persons, a significantly higher morbidity is associated to the procedure largely due to the necessity for extra-corporeal circulation. Further, open heart surgery is often poorly tolerated in elderly patients.

Thus if the extra-corporeal component of the procedure could be eliminated, morbidities and cost of valve replacement therapies would be significantly reduced.

While replacement of the aortic valve in a transcatheter manner is the subject of intense investigation, lesser attention has been focused on the mitral valve. This is in part reflective of the greater level of complexity associated to the native mitral valve apparatus and thus a greater level of difficulty with regards to inserting and anchoring the replacement prosthesis.

Several designs for catheter-deployed (transcatheter) aortic valve replacement are under various stages of development. The Edwards SAPIEN transcatheter heart valve is currently undergoing clinical trial in patients with calcific aortic valve disease who are considered high-risk for conventional open-heart valve surgery. This valve is deployable via a retrograde transarterial (transfemoral) approach or an antegrade transapical (transventricular) approach. A key aspect of the Edwards SAPIEN and other transcatheter aortic valve replacement designs is their dependence on lateral fixation (e.g. tines) that engages the valve tissues as the primary anchoring mechanism. Such a design basically relies on circumferential friction around the valve housing or stent to prevent dislodgement during the cardiac cycle. This anchoring mechanism is facilitated by, and may somewhat depend on, a calcified aortic valve annulus. This design also requires that the valve housing or stent have a certain degree of rigidity.

At least one transcatheter mitral valve design is currently in development. The Endovalve uses a folding tripod-like design that delivers a tri-leaflet bioprosthetic valve. It is designed to be deployed from a minimally invasive transatrial approach, and could eventually be adapted to a transvenous atrial septotomy delivery. This design uses “proprietary gripping features” designed to engage the valve annulus and leaflets tissues. Thus the anchoring mechanism of this device is essentially equivalent to that used by transcatheter aortic valve replacement designs.

Various problems continue to exist in this field, including problems with insufficient articulation and sealing of the valve within the native annulus, pulmonary edema due to poor atrial drainage, perivalvular leaking around the install prosthetic valve, lack of a good fit for the prosthetic valve within the native mitral annulus, atrial tissue erosion, excess wear on the nitinol structures, interference with the aorta at the posterior side of the mitral annulus, and lack of customization, to name a few. Accordingly, there is still a need for an improved valve having a commissural sealing structure for a prosthetic mitral valve.

### BRIEF SUMMARY OF THE INVENTION

The present invention relates to a self-expanding pre-configured compressible transcatheter prosthetic cardiovascular valve that comprises an atrial thrombogenic sealing mesh pocket cover disposed between a self-expanding inner wire frame and a self-expanding annular tissue-covered outer wire frame, said inner wire frame having articulating leaflets that define a valve function, said inner wire frame is disposed within the outer wire frame, said outer wire frame having an articulating circumferential collar, forming a multi-component prosthetic valve assembly for anchoring within the mitral valve or triscuspid valve of the heart.

Additional preferred embodiments include a tether structure connected to the inner wire frame or the outer wire frame, connected at a distal end to the inner wire frame or the outer wire frame and an epicardial pad connected to the proximal end of the tether structure.

In one preferred embodiment, the inner wire frame and the outer wire frame are made of a self-expanding compressible nickel-titanium biocompatible alloy.

In one preferred embodiment, the tissue is derived from adult, 90-day old, or 30 day old, bovine, ovine, equine or porcine pericardium, or from animal small intestine submucosa.

In one preferred embodiment, the tissue is synthetic material and is selected from the group consisting of polyester, polyurethane, and polytetrafluoroethylene.

In one preferred embodiment, the stabilized tissue or synthetic material is treated with anticoagulant.

In one preferred embodiment, the invention further comprises one or more standard anchoring elements, including but not limited to barbs, pins, and/or hooks, or combinations thereof to mount the valve within the cardiovascular valve annulus.

The invention also includes a method of treating a disease or disorder of a heart valve in a patient, which comprises the step of surgically deploying the prosthetic heart valve into the native annulus of the heart valve of the patient.

In one preferred embodiment, the native annulus is the mitral valve annulus or the tricuspid valve annulus.

In one preferred embodiment, the prosthetic heart valve is deployed by directly accessing the heart through the intercostal space, using an apical approach to enter the ventricle, and deploying the prosthetic heart valve into the native annulus using a catheter delivery system, or the prosthetic heart valve is deployed by directly accessing the heart through a thoracotomy, sternotomy, or minimally-invasive thoracic, thorascopic, or trans-diaphragmatic approach to enter the ventricle, or the prosthetic heart valve is deployed by directly accessing the heart through the intercostal space, using an approach through the lateral ventricular wall to enter the left ventricle, or the prosthetic heart valve is deployed by accessing the left atrium of the heart using a transvenous atrial septostomy approach, or the prosthetic heart valve is deployed by accessing the left ventricle of the heart using a transarterial retrograde aortic valve approach, or the prosthetic heart valve is deployed by accessing the left ventricle of the heart using a transvenous ventricular septostomy approach.

In another preferred embodiment, the method further comprises tethering the prosthetic heart valve to tissue within the left ventricle, or wherein the prosthetic heart valve is tethered to the apex of the ventricle using an epicardial tether securing device.

The design as provided focuses on the deployment of a pre-configured compressible transcatheter prosthetic cardiovascular valve which comprises the self-expanding wire frame mounted as an inner valve component within a outer mitral annulus collar component, with deployment via a minimally invasive surgical procedure utilizing the intercostal or subxyphoid space for valve introduction, but may also include standard retrograde, or antegrade transcatheter approaches. In order to accomplish this, the valve is formed in such a manner that it is self-expanding and is compressed to fit within a delivery system and secondarily ejected from the delivery system into the target location, for example the mitral or tricuspid valve annulus.

### BRIEF DESCRIPTION OF THE DRAWINGS

FIG. 1 is a top view of one embodiment of the present invention showing the atrial thrombogenic sealing pocket, and mesh pocket cover.

FIG. 2 is a perspective view of one embodiment of the present invention showing the atrial thrombogenic sealing pocket.

FIG. 3 is a second perspective view one embodiment of the present invention showing the atrial thrombogenic sealing pocket.

FIG. 4 is an exploded view of one embodiment of the subcomponent parts of the invention.

### DETAILED DESCRIPTION OF THE INVENTION

### Functions of the Atrial Thrombogenic Sealing Pocket

The atrial thrombogenic sealing pocket functions by providing a sealing mechanism between the inner wire frame and the outer wire frame that consists of a covered pocket for trapping or slowing blood with the goal of reducing hemodynamic washout and increasing thrombogenic sealing against perivalvular leakage.

Providing a valve having two wire frame components, an inner wire frame and an outer wire frame, allows for a space or gap to be created between them. Where the inner wire frame and outer wire frame are sewn on the lower or ventricular side, a pocket is formed facing the atrium. Covering this pocket with a permeable fabric or tissue allows blood flowing from the atrium to the ventricle to pool into the circumferential pocket area. The permeable covering prevents the pocket from being subjected to vigorous washout as the heart completes its cyclical transitions from diastole to systole and back. This wash-out phenomenon reduces and may preclude adequate clotting and/or ingrowth. For a prosthetic valve of this type, the strategies of promoting active in-growth and of using planned thrombogenesis on or near the implanted valve to address perivalvular leakage can be critical to the success of the valve. By covering the circumferential pocket between the inner and outer wire frames, blood is allowed to pool and/or commence thrombogenesis, reducing the incidence of perivalvular leaking compared to valves that do not utilize this strategy.

The pocket is formed by the joining, on one side, of the tissue attached to the cylindrical inner wire frame, and the tissue or fabric material attached to the outer annular wire frame. By joining in this fashion, a circumferential V-shaped or U-shaped pocket is formed.

In one embodiment, the cylindrical inner wire frame may extend into the atrium beyond the plane of the native annulus or atrial floor.

Accordingly, by providing an atrial thrombogenic sealing pocket or ring structure that is raised above the plane of the native annulus or atrial floor, complete leaflet coaptation is encouraged. During ventricular contraction or systole, the blood is ejected towards aortic valve to exit the heart but is also ejected retrograde towards the prosthetic mitral valve, which needs to remain closed during systole. Retrograde blood hitting the prosthetic valve leaflets cause the leaflets to close, preventing regurgitation into the left atrium. During diastole or ventricular filling, the blood needs to flow from the atrium into the ventricle without obstruction. However, when prosthetic leaflets are not properly placed or properly aligned, the leaflets can obstruct efficient filling of the ventricle or cause uneven ventricular output. During systole or atrial filling and ventricular ejection, sealing is important to avoid unwanted regurgitation or valvular leakage.

### Leaflet and Assembly Structure

The valve leaflets are held by, or within, a leaflet assembly. In one preferred embodiment of the invention, the leaflet assembly comprises a leaflet wire support structure to which the leaflets are attached and the entire leaflet assembly is housed within the stent body. In this embodiment, the assembly is constructed of wire and stabilized tissue to form a suitable platform for attaching the leaflets. In this aspect, the wire and stabilized tissue allow for the leaflet structure to be compressed when the prosthetic valve is compressed within the deployment catheter, and to spring open into the proper functional shape when the prosthetic valve is opened during deployment. In this embodiment, the leaflet assembly may optionally be attached to and housed within a separate cylindrical liner made of stabilized tissue or material, and the liner is then attached to line the interior of the stent body.

In this embodiment, the leaflet wire support structure is constructed to have a collapsible/expandable geometry. In a preferred embodiment, the structure is a single piece of wire. The wireform is, in one embodiment, constructed from a shape memory alloy such as Nitinol. The structure may optionally be made of a plurality of wires, including between 2 to 10 wires. Further, the geometry of the wire form is without limitation, and may optionally be a series of parabolic inverted collapsible arches to mimic the saddle-like shape of the native annulus when the leaflets are attached. Alternatively, it may optionally be constructed as collapsible concentric rings, or other similar geometric forms that are able to collapse/compress which is followed by an expansion to its functional shape. In certain preferred embodiments, there may be 2, 3 or 4 arches. In another embodiment, closed circular or ellipsoid structure designs are contemplated. In another embodiment, the wire form may be an umbrella-type structure, or other similar unfold-and-lock-open designs. A preferred embodiment utilizes super elastic Nitinol wire approximately 0.015″ in diameter. In this embodiment, the wire is wound around a shaping fixture in such a manner that 2-3 commissural posts are formed. The fixture containing the wrapped wire is placed in a muffle furnace at a pre-determined temperature to set the shape of the wire form and to impart it's super elastic properties. Secondarily, the loose ends of the wireform are joined with a stainless steel or Nitinol tube and crimped to form a continuous shape. In another preferred embodiment, the commissural posts of the wireform are adjoined at their tips by a circular connecting ring, or halo, whose purpose is to minimize inward deflection of the post(s).

In another preferred embodiment, the leaflet assembly is constructed solely of stabilized tissue or other suitable material without a separate wire support structure. The leaflet assembly in this embodiment is also disposed within the lumen of the stent and is attached to the stent to provide a sealed joint between the leaflet assembly and the inner wall of the stent. By definition, it is contemplated within the scope of the invention that any structure made from stabilized tissue and/or wire(s) related to supporting the leaflets within the stent constitute a leaflet assembly.

In this embodiment, stabilized tissue or suitable material may also optionally be used as a liner for the inner wall of the stent and is considered part of the leaflet assembly.

Liner tissue or biocompatible material may be processed to have the same or different mechanical qualities, e.g. thickness, durability, etc. from the leaflet tissue.

### Functions of the Collar or Flared End of the Stent to Effect Atrial Sealing

The flared collar-end, also known as a collar or cuff, functions in a variety of ways. The first function of the flared end or cuff is to inhibit perivalvular leak/regurgitation of blood around the prosthesis. By flexing and sealing across the irregular contours of the annulus and atrium, leaking is minimized and/or prevented.

The second function of the flared end or cuff is to provide an adjustable and/or compliant bioprosthetic valve. The heart and its structures undergo complex conformational changes during the cardiac cycle. For example, the mitral valve annulus has a complex geometric shape known as a hyperbolic parabloid much like a saddle, with the horn being anterior, the seat back being posterior, and the left and right valleys located medially and laterally. Beyond this complexity, the area of the mitral annulus changes over the course of the cardiac cycle. Further, the geometry of the tricuspid valve and tricuspid annulus continues to be a topic of research, posing its own particular problems. Accordingly, compliance is a very important but unfortunately often overlooked requirement of cardiac devices. Compliance here refers to the ability of the valve to maintain structural position and integrity during the cardiac cycle. Compliance with the motion of the heart is a particularly important feature, especially the ability to provide localized compliance where the underlying surfaces are acting differently from the adjacent surfaces. This ability to vary throughout the cardiac cycle allows the valve to remain seated and properly deployed in a manner not heretofore provided.

Additionally, compliance may be achieved through the use of the tethers where the tethers are preferably made from an elastic material. Tether-based compliance may be used alone, or in combination with the flared end or cuff-based compliance.

The third function of the flared end or cuff and valve is to provide a valve that, during surgery, is able to be seated and be able to contour to the irregular surfaces of the atrium. The use of independent tethers allows for side to side fitting of the valve within the annulus. For example, where three tethers are used, they are located circumferentially about 120 degrees relative to each other which allows the surgeon to observe whether or where perivalvular leaking might be occurring and to pull on one side or the other to create localized pressure and reduce or eliminate the leaking.

The fourth function of the flared end or cuff is to counter the forces that act to displace the prosthesis toward/into the ventricle (i.e. atrial pressure and flow-generated shear stress) during ventricular filling.

Additional features of the flared end or cuff include that it functions to strengthen the leaflet assembly/stent combination by providing additional structure. Further, during deployment, the flared end or cuff functions to guide the entire structure, the prosthetic valve, into place at the mitral annulus during deployment and to keep the valve in place once it is deployed. Another important function is to reduce pulmonary edema by improving atrial drainage.

### Flared End or Cuff Structure

The flared end or cuff is a substantially flat plate that projects beyond the diameter of the tubular stent to form a rim or border. As used herein, the term flared end, cuff, flange, collar, bonnet, apron, or skirting are considered to be functionally equivalent. When the tubular stent is pulled through the mitral valve aperture, the mitral annulus, by the tether loops in the direction of the left ventricle, the flared end or cuff acts as a collar to stop the tubular stent from traveling any further through the mitral valve aperture. The entire prosthetic valve is held by longitudinal forces between the flared end or cuff which is seated in the left atrium and mitral annulus, and the ventricular tethers attached to the left ventricle.

The flared end or cuff is formed from a stiff, flexible shape-memory material such as the nickel-titanium alloy material Nitinol TM wire that is covered by stabilized tissue or other suitable biocompatible or synthetic material. In one embodiment, the flared end or cuff wire form is constructed from independent articulating radial tines or posts of wire extending axially around the circumference of the bend or seam where the flared end or cuff transitions to the tubular stent (in an integral flared end or cuff) or where the flared end or cuff is attached to the stent (where they are separate, but joined components).

Once covered by stabilized tissue or material, the articulating radial tines or posts of wire provide the flared end or cuff the ability to travel up and down, to articulate, along the longitudinal axis that runs through the center of the tubular stent. In other words, the individual articulating radial tines or posts of wire can independently move up and down, and can spring back to their original position due to the relative stiffness of the wire. The tissue or material that covers the flared end or cuff wire has a certain modulus of elasticity such that, when attached to the wire of the flared end or cuff, is able to allow the wire spindles to move. This flexibility gives the flared end or cuff, upon being deployed within a patient's heart, the ability to conform to the anatomical shape necessary for a particular application. In the example of a prosthetic mitral valve, the flared end or cuff is able to conform to the irregularities of the left atrium and shape of the mitral annulus, and to provide a tight seal against the atrial tissue adjacent the mitral annulus and the tissue within the mitral annulus. As stated previously, this feature importantly provides a degree of flexibility in sizing the a mitral valve and prevents blood from leaking around the implanted prosthetic heart valve.

An additional important aspect of the flared end or cuff dimension and shape is that, when fully seated and secured, the edge of the flared end or cuff preferably should not be oriented laterally into the atrial wall such that it can produce a penetrating or cutting action on the atrial wall.

In one preferred embodiment, the wire spindles of the flared end or cuff are substantially uniform in shape and size. In another preferred embodiment of the present invention, each loop or spindle may be of varying shapes and sizes. In this example, it is contemplated that the articulating radial tines or posts of wire may form a pattern of alternating large and small articulating radial tines or posts of wire, depending on where the valve is being deployed. In the case of a prosthetic mitral valve, pre-operative imaging may allow for customizing the structure of the flared end or cuff depending on a particular patient's anatomical geometry in the vicinity of the mitral annulus.

The flared end or cuff wire form is constructed so as to provide sufficient structural integrity to withstand the intracardiac forces without collapsing. The flared end or cuff wire form is preferably constructed of a superelastic metal, such as Nitinol™® and is capable of maintaining its function as a sealing collar for the tubular stent while under longitudinal forces that might cause a structural deformation or valve displacement. It is contemplated as within the scope of the invention to optionally use other shape memory alloys such as Cu—Zn—Al—Ni alloys, and Cu—Al—Ni alloys. The heart is known to generate an average left atrial pressure between about 8 and 30 mm Hg (about 0.15 to 0.6 psi). This left atrial filling pressure is the expected approximate pressure that would be exerted in the direction of the left ventricle when the prosthesis is open against the outer face of the flared end or cuff as an anchoring force holding the flared end or cuff against the atrial tissue that is adjacent the mitral valve. The flared end or cuff counteracts this longitudinal pressure against the prosthesis in the direction of the left ventricle to keep the valve from being displaced or slipping into the ventricle. In contrast, left ventricular systolic pressure, normally about 120 mm Hg, exerts a force on the closed prosthesis in the direction of the left atrium. The tethers counteract this force and are used to maintain the valve position and withstand the ventricular force during ventricular contraction or systole. Accordingly, the flared end or cuff has sufficient structural integrity to provide the necessary tension against the tethers without being dislodged and pulled into the left ventricle. After a period of time, changes in the geometry of the heart and/or fibrous adhesion between prosthesis and surrounding cardiac tissues may assist or replace the function of the ventricular tethers in resisting longitudinal forces on the valve prosthesis during ventricular contraction.

### Wire Form Structures

Preferably, superelastic metal wire, such as Nitinol™ wire, is used for the outer wire form or stent, for the inner wire-based leaflet assembly that is disposed within the outer stent, and for the flared end or cuff wire form. As stated, it is contemplated as within the scope of the invention to optionally use other shape memory alloys such as Cu—Zn—Al—Ni alloys, and Cu—Al—Ni alloys. It is contemplated that the stent may be constructed as a braided stent or as a laser cut stent. Such stents are available from any number of commercial manufacturers, such as Pulse Systems. Laser cut stents are preferably made from Nickel-Titanium (Nitinol™), but also without limitation made from stainless steel, cobalt chromium, titanium, and other functionally equivalent metals and alloys, or Pulse Systems braided stent that is shape-set by heat treating on a fixture or mandrel.

One key aspect of the stent design is that it be compressible and when released have the stated property that it return to its original (uncompressed) shape. This requirement limits the potential material selections to metals and plastics that have shape memory properties. With regards to metals, Nitinol has been found to be especially useful since it can be processed to be austhenitic, martensitic or super elastic. Martensitic and super elastic alloys can be processed to demonstrate the required compression features.

### Laser Cut Stent

One possible construction of the stent envisions the laser cutting of a thin, isodiametric Nitinol tube. The laser cuts form regular cutouts in the thin Nitinol tube. Secondarily the tube is placed on a mold of the desired shape, heated to the Martensitic temperature and quenched. The treatment of the stent in this manner will form a stent or stent/flared end or cuff that has shape memory properties and will readily revert to the memory shape at the calibrated temperature.

### Braided Wire Stent

A stent can be constructed utilizing simple braiding techniques. Using a Nitinol wire—for example a 0.012″ wire—and a simple braiding fixture, the wire is wound on the braiding fixture in a simple over/under braiding pattern until an isodiametric tube is formed from a single wire. The two loose ends of the wire are coupled using a stainless steel or Nitinol coupling tube into which the loose ends are placed and crimped. Angular braids of approximately 60 degrees have been found to be particularly useful. Secondarily, the braided stent is placed on a shaping fixture and placed in a muffle furnace at a specified temperature to set the stent to the desired shape and to develop the martensitic or super elastic properties desired.

The stent as envisioned in one preferred embodiment is designed such that the ventricular aspect of the stent comes to 2-5 points onto which anchoring sutures are affixed. The anchoring sutures (tethers) will traverse the ventricle and ultimately be anchored to the epicardial surface of the heart approximately at the level of the apex. The tethers when installed under slight tension will serve to hold the valve in place, i e inhibit paravalvular leakage during systole.

Deployment within the Valvular Annulus

The prosthetic heart valve is, in one embodiment, apically delivered through the apex of the left ventricle of the heart using a catheter system. In one aspect of the apical delivery, the catheter system accesses the heart and pericardial space by intercostal delivery. In another delivery approach, the catheter system delivers the prosthetic heart valve using either an antegrade or retrograde delivery approach using a flexible catheter system, and without requiring the rigid tube system commonly used. In another embodiment, the catheter system accesses the heart via a trans-septal approach.

In one non-limiting preferred embodiment, the stent body extends into the ventricle about to the edge of the open mitral valve leaflets (approximately 25% of the distance between the annulus and the ventricular apex). The open native leaflets lay against the outside stent wall and parallel to the long axis of the stent (i.e. the stent holds the native mitral valve open).

In one non-limiting preferred embodiment, the diameter should approximately match the diameter of the mitral annulus. Optionally, the valve may be positioned to sit in the mitral annulus at a slight angle directed away from the aortic valve such that it is not obstructing flow through the aortic valve. Optionally, the outflow portion (bottom) of the stent should not be too close to the lateral wall of the ventricle or papillary muscle as this position may interfere with flow through the prosthesis. As these options relate to the tricuspid, the position of the tricuspid valve may be very similar to that of the mitral valve.

In another embodiment, the prosthetic valve is sized and configured for use in areas other than the mitral annulus, including, without limitation, the tricuspid valve between the right atrium and right ventricle. Alternative embodiments may optionally include variations to the flared end or cuff structure to accommodate deployment to the pulmonary valve between the right ventricle and pulmonary artery, and the aortic valve between the left ventricle and the aorta. In one embodiment, the prosthetic valve is optionally used as a venous backflow valve for the venous system, including without limitation the vena cava, femoral, subclavian, pulmonary, hepatic, renal and cardiac. In this aspect, the flared end or cuff feature is utilized to provide additional protection against leaking.

### Tethers

In one preferred embodiment, there are tethers attached to the prosthetic heart valve that extend to one or more tissue anchor locations within the heart. In one preferred embodiment, the tethers extend downward through the left ventricle, exiting the left ventricle at the apex of the heart to be fastened on the epicardial surface outside of the heart. Similar anchoring is contemplated herein as it regards the tricuspid, or other valve structure requiring a prosthetic. There may be from 1 to 8 tethers which are preferably attached to the stent.

In another preferred embodiment, the tethers may optionally be attached to the flared end or cuff to provide additional control over position, adjustment, and compliance. In this preferred embodiment, one or more tethers are optionally attached to the flared end or cuff, in addition to, or optionally, in place of, the tethers attached to the stent. By attaching to the flared end or cuff and/or the stent, an even higher degree of control over positioning, adjustment, and compliance is provided to the operator during deployment.

During deployment, the operator is able to adjust or customize the tethers to the correct length for a particular patient's anatomy. The tethers also allow the operator to tighten the flared end or cuff onto the tissue around the valvular annulus by pulling the tethers, which creates a leak-free seal.

In another preferred embodiment, the tethers are optionally anchored to other tissue locations depending on the particular application of the prosthetic heart valve. In the case of a mitral valve, or the tricuspid valve, there are optionally one or more tethers anchored to one or both papillary muscles, septum, and/or ventricular wall.

The tethers, in conjunction with the flared end or cuff, provide for a compliant valve which has heretofore not been available. The tethers are made from surgical-grade materials such as biocompatible polymer suture material. Non-limiting examples of such material include ultra high-molecular weight polyethylene (UHMWPE), 2-0 exPFTE (polytetrafluoroethylene) or 2-0 polypropylene. In one embodiment the tethers are inelastic. It is also contemplated that one or more of the tethers may optionally be elastic to provide an even further degree of compliance of the valve during the cardiac cycle. Upon being drawn to and through the apex of the heart, the tethers may be fastened by a suitable mechanism such as tying off to a pledget or similar adjustable button-type anchoring device to inhibit retraction of the tether back into the ventricle. It is also contemplated that the tethers might be bioresorbable/bioabsorbable and thereby provide temporary fixation until other types of fixation take hold such a biological fibrous adhesion between the tissues and prosthesis and/or radial compression from a reduction in the degree of heart chamber dilation.

Further, it is contemplated that the prosthetic heart valve may optionally be deployed with a combination of installation tethers and permanent tethers, attached to either the stent or flared end or cuff, or both, the installation tethers being removed after the valve is successfully deployed. It is also contemplated that combinations of inelastic and elastic tethers may optionally be used for deployment and to provide structural and positional compliance of the valve during the cardiac cycle.

### Pledget

In one embodiment, to control the potential tearing of tissue at the apical entry point of the delivery system, a circular, semi-circular, or multi-part pledget is employed. The pledget may be constructed from a semi-rigid material such as PFTE felt. Prior to puncturing of the apex by the delivery system, the felt is firmly attached to the heart such that the apex is centrally located. Secondarily, the delivery system is introduced through the central area, or orifice as it may be, of the pledget. Positioned and attached in this manner, the pledget acts to control any potential tearing at the apex.

### Tines/Barbs

In another embodiment the valve can be seated within the valvular annulus through the use of tines or barbs. These may be used in conjunction with, or in place of one or more tethers. The tines or barbs are located to provide attachment to adjacent tissue. In one preferred embodiment, the tines are optionally circumferentially located around the bend/transition area between the stent and the flared end or cuff. Such tines are forced into the annular tissue by mechanical means such as using a balloon catheter. In one non-limiting embodiment, the tines may optionally be semi-circular hooks that upon expansion of the stent body, pierce, rotate into, and hold annular tissue securely.

### Stabilized Tissue or Biocompatible Material

In one embodiment, it is contemplated that multiple types of tissue and biocompatible material may be used to cover the flared end or cuff,to form the valve leaflets, to form a wireless leaflet assembly, and/or to line both the inner and/or outer lateral walls of the stent. As stated previously, the leaflet component may be constructed solely from stabilized tissue, without using wire, to create a leaflet assembly and valve leaflets. In this aspect, the tissue-only leaflet component may be attached to the stent with or without the use of the wire form. In a preferred embodiment, there can be anywhere from 1, 2, 3 or 4 leaflets, or valve cusps.

It is contemplated that the tissue may be used to cover the inside of the stent body, the outside of the stent body, and the top and/or bottom side of the flared end or cuff wire form, or any combination thereof.

In one preferred embodiment, the tissue used herein is optionally a biological tissue and may be a chemically stabilized valve of an animal, such as a pig. In another preferred embodiment, the biological tissue is used to make leaflets that are sewn or attached to a metal frame. This tissue is chemically stabilized pericardial tissue of an animal, such as a cow (bovine pericardium) or sheep (ovine pericardium) or pig (porcine pericardium) or horse (equine pericardium).

Preferably, the tissue is bovine pericardial tissue. Examples of suitable tissue include that used in the products Duraguard®, Peri-Guard®, and Vascu-Guard®, all products currently used in surgical procedures, and which are marketed as being harvested generally from cattle less than 30 months old. Other patents and publications disclose the surgical use of harvested, biocompatible animal thin tissues suitable herein as biocompatible “jackets” or sleeves for implantable stents, including for example, U.S. Pat. No. 5,554,185 to Block, U.S. Pat. No. 7,108,717 to Design & Performance-Cyprus Limited disclosing a covered stent assembly, U.S. Pat. No. 6,440,164 to Scimed Life Systems, Inc. disclosing a bioprosthetic valve for implantation, and U.S. Pat. No. 5,336,616 to LifeCell Corporation discloses acellular collagen-based tissue matrix for transplantation.

In one preferred embodiment, the valve leaflets may optionally be made from a synthetic material such a polyurethane or polytetrafluoroethylene. Where a thin, durable synthetic material is contemplated, e.g. for covering the flared end or cuff, synthetic polymer materials such expanded polytetrafluoroethylene or polyester may optionally be used. Other suitable materials may optionally include thermoplastic polycarbonate urethane, polyether urethane, segmented polyether urethane, silicone polyether urethane, silicone-polycarbonate urethane, and ultra-high molecular weight polyethylene. Additional biocompatible polymers may optionally include polyolefins, elastomers, polyethylene-glycols, polyethersulphones, polysulphones, polyvinylpyrrolidones, polyvinylchlorides, other fluoropolymers, silicone polyesters, siloxane polymers and/or oligomers, and/or polylactones, and block co-polymers using the same.

In another embodiment, the valve leaflets may optionally have a surface that has been treated with (or reacted with) an anti-coagulant, such as, without limitation, immobilized heparin. Such currently available heparinized polymers are known and available to a person of ordinary skill in the art.

Alternatively, the valve leaflets may optionally be made from pericardial tissue or small intestine submucosal tissue.

### Description of Figures

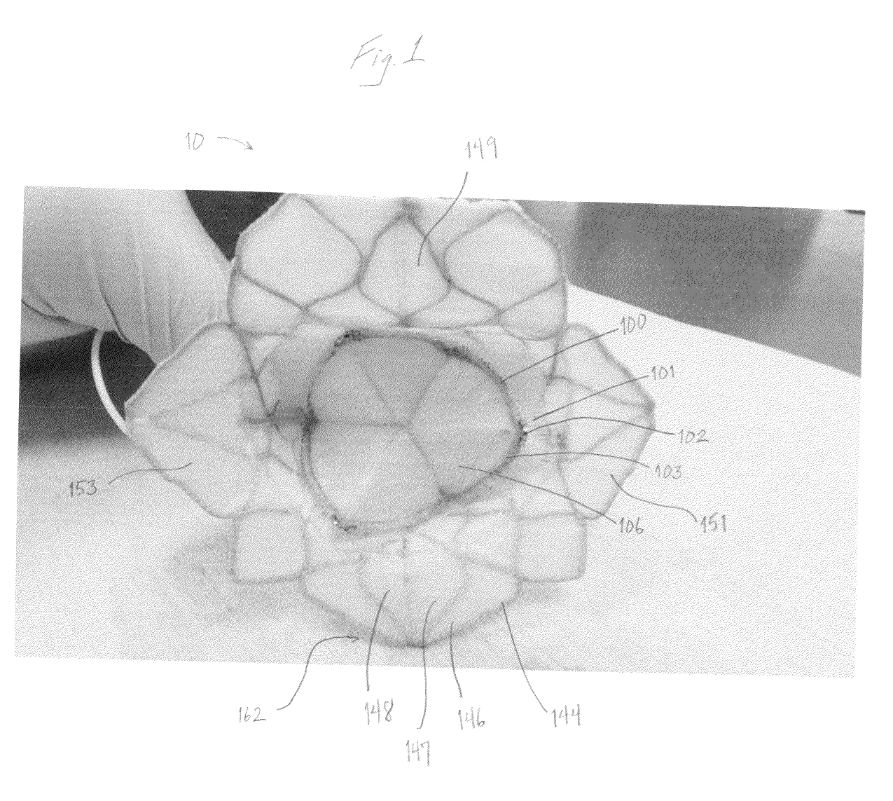

Referring now to the FIGURES, FIG. 1 is a top-view of a self-expanding pre-configured compressible transcatheter prosthetic cardiovascular valve 10 contemplated herein, that contains as a sub-component, a self-expanding inner wire frame 100 with atrial thrombogenic sealing pocket cover 101. In this valve 10, the inner wire frame 100 has an atrial thrombogenic sealing pocket cover 101 formed from a circular piece of wire, or halo 103, with a permeable mesh fabric or tissue, that is sewn and thereby connected to the inner wire frame 100 and to the leaflet tissue 106 that forms part of the leaflet assembly. The inner wire frame 100 forms an inner wireframe structure made of nitinol wire that supports leaflet tissue 106 sewn to the inner wire frame and functions as a valve. The inner wire frame 100 in FIG. 1 is composed of three U-shaped wire components joined at their opened ends to form junctions 102. Leaflet tissue 106 is sewn to these components to form articulating leaflets 106 creating and functioning as a prosthetic tricuspid valve.

The inner wireframe 100 also has (tether) attachment apertures 111 (not shown), for attaching tether structure 160 (not shown). Tether 160 is connected to epicardial securing pad 154 (not shown).

In operation, the inner wireframe 100 (with internal leaflet 106), is disposed within and secured within the outer stent/wire frame 144. Outer stent/wire frame 144 may also have in various embodiments an outer stent tissue material such as is illustrated as 150. Outer stent/wire frame 144 has an articulating collar 146 which has a collar cover 148. Articulating collar 146 is specifically shaped to solve leakage issues arising from native structures. In particular, collar 146 is composed of an A2 segment 147, a P2 segment 149, and two commissural segments, the A1-P1 segment 151, and the A3-P3 segment 153. The collar 146 may also have in preferred embodiments a shortened or flattened or D-shaped section 162 of the A2 segment in order to accommodate and solve left ventricular outflow tract (LVOT) obstruction issues.

In operation, the valve 10 may be deployed as a prosthetic mitral valve using catheter delivery techniques. The entire valve 10 is compressed within a narrow catheter and delivered to the annular region of the native valve, preferably the left atrium, with a pre-attached tether apparatus. There, the valve 10 is pushed out of the catheter where it springs open into its pre-formed functional shape without the need for manual expansion using an inner balloon catheter. When the valve 10 is pulled into place, the outer stent 144 is seated in the native mitral annulus, leaving the articulating collar 146 to engage the atrial floor and prevent pull-thru (where the valve is pulled into the ventricle). The native leaflets are not cut-away as has been taught in prior prosthetic efforts, but are used to provide a tensioning and sealing function around the outer stent 144. The valve 10 must be asymmetrically deployed in order to address LVOT problems where non-accommodating prosthetic valves push against the A2 anterior segment of the mitral valve and close blood flow through the aorta, which anatomically sits immediately behind the A2 segment of the mitral annulus. Thus, D-shaped section 162 is deployed immediately adjacent/contacting the A2 segment since the flattened D-shaped section 162 is structurally smaller and has a more vertical profile (closer to paralleling the longitudinal axis of the outer stent) and thereby provides less pressure on the A2 segment. Once the valve 10 is properly seated, tether 160 may be extended out through the apical region of the left ventricle and secured using an epicardial pad 154 or similar suture-locking attachment mechanism (not shown).

In an alternate embodiment, the tether attachment structures are on the outer stent 144, which would then have (tether) attachment apertures 113 for attaching tether structure 160 to epicardial securing pad 154.

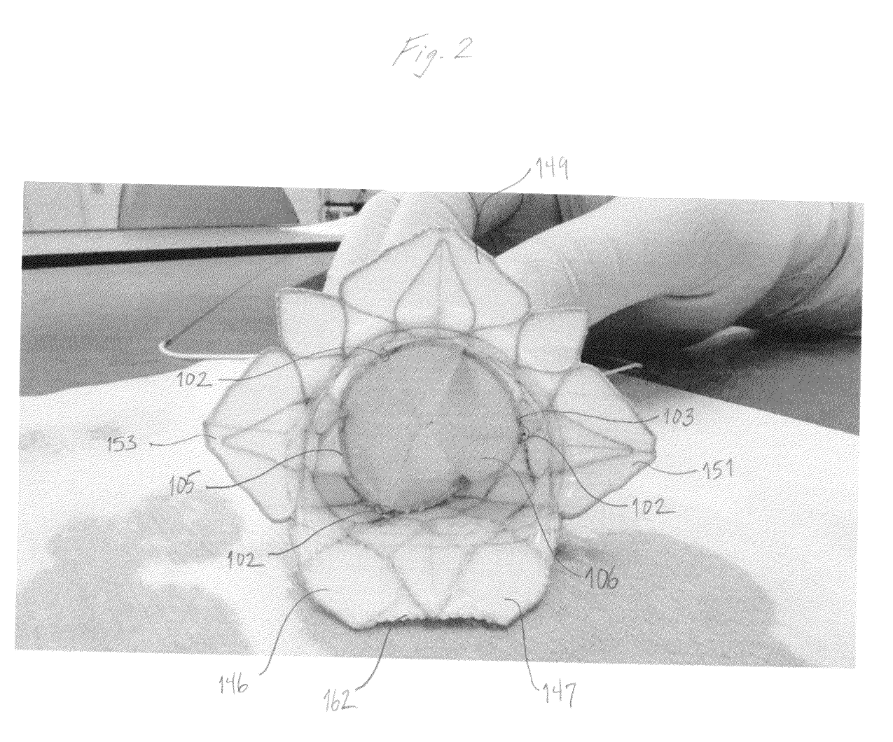

FIG. 2 is a top, or atrial, view of an embodiment of the valve before the mesh tissue or fabric pocket covering has been added. FIG. 2 also shows where the halo 103 is not formed at or above the plane of the collar or of the atrial floor, all of which are contemplated as within the scope of the invention herein. Rather, halo 103 in this comparative example is formed lower within the outer wire frame and is seated below the plane of the collar or of the atrial floor. Thus reducing or eliminating the beneficial effects of thrombogenic sealing. However, FIG. 2 also shows that a small pocket 105 is nonetheless formed through the use of an inner wire frame and outer wire frame.

FIG. 2 shows the top of the junction tip 102 of the three U-shaped wire components of inner wire frame 100 joined at their opened ends to form junctions 102. Leaflet tissue 106 is sewn to these components to form articulating leaflets 106 creating and functioning as a prosthetic tricuspid valve. Atrial Thrombogenic Sealing Pocket 105 is shown below the plane of the collar. FIG. 2 shows vertical A2 segment 147, the P2 segment 149, and the commissural A1-P1 segment 151 an A3-P3 segment 153. FIG. 2 shows how upon deployment blood would fill the void or gap 105 between the inner 100 and outer wire frame 144 of the valve 10. This blood creates a temporary fluid seal that pools in that space and provide a pressure buffer against the leakage inducing forces that accompany systolic and diastolic related intra-atrial and intra-ventricular pressure. FIG. 2 also provides a good illustration of collar 146 that may also have in preferred embodiments a shortened or flattened or D-shaped section 162 of the A2 segment in order to accommodate and solve left ventricular outflow tract (LVOT) obstruction issues.

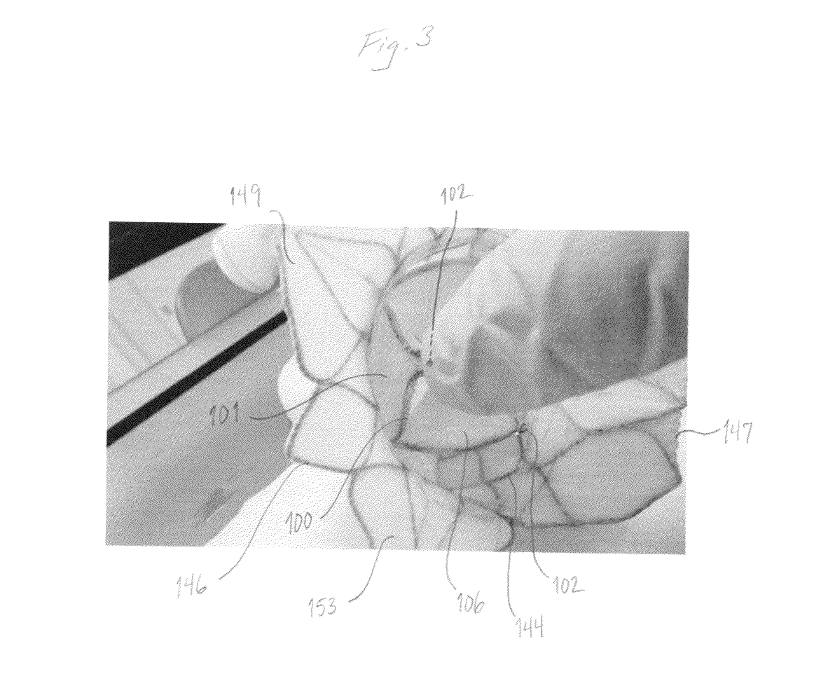

FIG. 3 is a perspective side view of the P2 area 149 and A3-P3 area 153 of a self-expanding pre-configured compressible transcatheter prosthetic cardiovascular valve 10 contemplated herein, that contains as a sub-component, a self-expanding inner wire frame 100 with atrial thrombogenic sealing pocket 101. FIG. 3 shows one of the three U-shaped wire components of inner wire frame 100 joined at their opened ends to form junctions 102. Leaflet tissue 106 is sewn to these components to form articulating leaflets 106 creating and functioning as a prosthetic tricuspid valve. Atrial Thrombogenic Sealing Pocket 101 is shown slightly below the plane of the majority of collar 146 except for the vertical A2 segment 147, the P2 segment 149, and the commissural A1-P1 segment 151 (not shown) and A3-P3 segment 153. FIG. 3 shows how upon deployment blood would fill the void or gap 101 between the inner wire frame 100 and outer wire frame 144 at the A3-P3 segment 153 area of the valve 10. This blood creates a temporary fluid seal that would pool in that space and provide a pressure buffer against the leakage inducing forces that accompany systolic and diastolic related intra-atrial and intra-ventricular pressure.

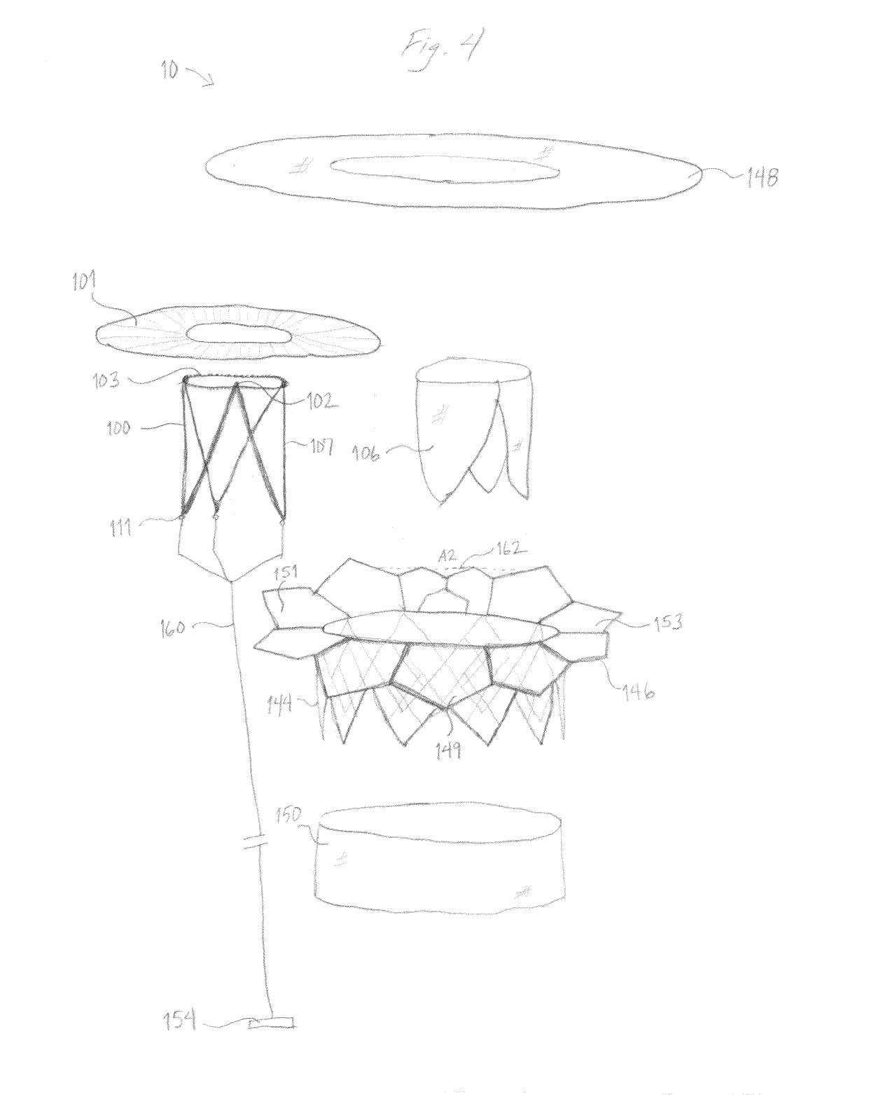

FIG. 4 is an exploded view of one embodiment of a pre-configured compressible transcatheter prosthetic cardiovascular valve 10 contemplated herein, that contains as a sub-component, a self-expanding wire frame 100 with atrial thrombogenic sealing pocket cover 101. The pocket itself is formed between inner wire frame leaflet tissue 106, as the inside of the V-shaped or U-shaped pocket, and the outer wire frame 144 with cover 150, as the outside of the V-shaped or U-shaped pocket. In this valve 10, the inner wire frame 100 has an atrial thrombogenic sealing pocket cover 101 formed from a circular piece of wire, or halo 103, with a permeable mesh fabric or tissue 101, that is sewn and thereby connected to the inner wire frame 100 and to the leaflet tissue 106 that forms part of the leaflet assembly. The inner wire frame 100 forms an inner wireframe structure made of nitinol wire that supports leaflet tissue 106 sewn to the inner wire frame and functions as a valve. The inner wire frame 100 is composed of three main U-shaped wire components 107 joined at their opened ends to form junctions 102. Additional wire cross-members or struts may also be part of the inner wire frame, but this is optional.

In this valve 10, the wire frame 100 is sewn with tissue 106 and acts a cover to prevent valvular leakage. The inner wireframe structure 100 contains the leaflet structure 106 comprised of articulating leaflets that define a valve function. The leaflet structure 106 is sewn to the inner wireframe 100. The wireframe 100 also has (tether) attachment apertures 111 for attaching tether structure 160. Tether 160 is shown in this example as connected to epicardial securing pad 154. In operation, the covered wireframe 100 (with internal leaflet 106), is disposed within and secured within the outer stent 144. Outer stent 144 may also have in various embodiments an outer stent cover such as is illustrated as 150. Outer stent 144 has an articulating collar 146 which has a collar cover 148. Articulating collar 146 may also have in preferred embodiments a flattened or D-shaped section 162 at the A2 area to accommodate and solve left ventricular outflow tract (LVOT) obstruction issues. Collar 146 may also have specially formed commissural segments to prevent commissural leakage at A1-P1 segment 151 and at A3-P3 segment 153. In operation, the valve 10 may be deployed as a prosthetic mitral valve using catheter delivery techniques. The entire valve 10 is compressed within a narrow catheter and delivered to the annular region of the native valve, preferably the left atrium, with a pre-attached tether apparatus. There, the valve 10 is pushed out of the catheter where it springs open into its pre-formed functional shape without the need for manual expansion using an inner balloon catheter. When the valve 10 is pulled into place, the outer stent 144 is seated in the native mitral annulus, leaving the articulating collar 146 to engage the atrial floor and prevent pull-thru (where the valve is pulled into the ventricle). The native leaflets are not cut-away as has been taught in prior prosthetic efforts, but are used to provide a tensioning and sealing function around the outer stent 144. The valve 10 must be asymmetrically deployed in order to address LVOT problems where non-accommodating prosthetic valves push against the A2 anterior segment of the mitral valve and close blood flow through the aorta, which anatomically sits immediately behind the A2 segment of the mitral annulus. Thus, D-shaped section 162 is deployed immediately adjacent/contacting the A2 segment since the flattened D-shaped section 162 is structurally smaller and has a more vertical profile (closer to paralleling the longitudinal axis of the outer stent) and thereby provides less pressure on the A2 segment. Once the valve 10 is properly seated, tether 160 may be extended out through the apical region of the left ventricle and secured using an epicardial pad 154 or similar suture-locking attachment mechanism.

The references recited herein are incorporated herein in their entirety, particularly as they relate to teaching the level of ordinary skill in this art and for any disclosure necessary for the commoner understanding of the subject matter of the claimed invention. It will be clear to a person of ordinary skill in the art that the above embodiments may be altered or that insubstantial changes may be made without departing from the scope of the invention. Accordingly, the scope of the invention is determined by the scope of the following claims and their equitable Equivalents.

What is claimed is:

1. A self-expanding pre-configured compressible transcatheter prosthetic cardiovascular valve that comprises an atrial thrombogenic sealing pocket cover disposed between a self-expanding inner wire frame and a self-expanding annular tissue-covered outer wire frame, said inner wire frame having articulating tissue leaflets that define a valve function, said inner wire frame is disposed within the outer wire frame, said outer wire frame having an articulating circumferential collar, forming a multi-component prosthetic valve assembly for anchoring within the mitral valve or triscuspid valve of the heart.

2. The valve of claim 1, further comprising a tether structure connected to the inner wire frame or the outer wire frame.

3. The valve of claim 1, further comprising a tether structure connected at a distal end to the inner wire frame or the outer wire frame and an epicardial pad connected to the proximal end of the tether structure.

4. The valve of claim 1, wherein the inner wire frame and the outer wire frame are made of a self-expanding compressible nickel-titanium biocompatible alloy.

5. The valve of claim 1, further comprising wherein the tissue is derived from adult, 90-day old, or 30 day old, bovine, ovine, equine or porcine pericardium, or from animal small intestine submucosa.

6. The valve of claim 1, further comprising wherein the cover or tissue is synthetic material and is selected from the group consisting of polyester, polyurethane, and polytetrafluoroethylene.

7. The valve of claim 1, wherein the cover or tissue is stabilized tissue or synthetic material is treated with anticoagulant.

8. The valve of claim 1, further comprising one or more standard anchoring elements, including but not limited to barbs, pins, and/or hooks, or combinations thereof to mount the valve within the cardiovascular valve annulus.

9. A method of treating a disease or disorder of a heart valve in a patient, which comprises the step of surgically deploying the prosthetic heart valve according to claim 1 into the native annulus of the heart valve of the patient.

10. The method of claim 9, wherein the native annulus is the mitral valve annulus or the tricuspid valve annulus.

11. The method of claim 9, wherein the prosthetic heart valve is deployed by directly accessing the heart through the intercostal space, using an apical approach to enter the ventricle, and deploying the prosthetic heart valve into the native annulus using a catheter delivery system.

12. The method of claim 9, wherein the prosthetic heart valve is deployed by directly accessing the heart through a thoracotomy, sternotomy, or minimally-invasive thoracic, thorascopic, or trans-diaphragmatic approach to enter the ventricle.

13. The method of claim 9, wherein the prosthetic heart valve is deployed by directly accessing the heart through the intercostal space, using an approach through the lateral ventricular wall to enter the left ventricle.

14. The method of claim 9, wherein the prosthetic heart valve is deployed by accessing the left atrium of the heart using a transvenous atrial septostomy approach.

15. The method of claim 9, wherein the prosthetic heart valve is deployed by accessing the left ventricle of the heart using a transarterial retrograde aortic valve approach.

16. The method of claim 9, wherein the prosthetic heart valve is deployed by accessing the left ventricle of the heart using a transvenous ventricular septostomy approach.

17. The method of any of claims 9-16, further comprising tethering the prosthetic heart valve to tissue within the left ventricle.

18. The method of any of claim 9-16, wherein the prosthetic heart valve is tethered to the apex of the ventricle using an epicardial tether securing device.
